(19) United States
(12) Patent Application Publication
(10) Pub. No.: US 2008/0226715 A1
(43) Pub. Date: Sep. 18, 2008

(54) THERAPEUTIC COMPOSITIONS AND METHODS

(76) Inventors: Albert Cha, Los Altos, CA (US); Anthony Joonkyoo Yun, Menlo Park, CA (US)

Correspondence Address: WILSON SONSINI GOODRICH & ROSATI, 650 PAGE MILL ROAD, PALO ALTO, CA, 94304-1050, US

(21) Appl. No.: 12/049,223

(22) Filed: Mar. 14, 2008

Related U.S. Application Data
(60) Provisional application No. 60/895,371, filed on Mar. 16, 2007.

(51) Int. Cl. A61K 9/22; A61K 31/437; A61P 25/06; A61P 25/16; A61K 31/55

(52) U.S. Cl. 424/468; 514/299; 514/217

(57) ABSTRACT

The present invention provides compositions, methods and kits for treating, preventing or reducing the risk of developing a CNS disorder. In general, the invention involves utilizing caffeine for preventing or alleviating pathological symptoms of a CNS disorder, such as headache, epilepsy, pain, Parkinson's disease, psychiatric disorders such as anxiety, bipolar disorder, depression, and schizophrenia, ADD, and ADHD.

### CROSS-REFERENCE

This application claims the benefit of U.S. Provisional Application No. 60/895,371, filed Mar. 16, 2007, which application is incorporated herein by reference

### FIELD OF THE INVENTION

The present invention relates to methods of treating or preventing central nervous system (CNS) disorders, such as headache, epilepsy, pain, Parkinson's disease, psychiatric disorders such as anxiety, bipolar disorder, depression, and schizophrenia, attention deficit disorders (ADD), and attention deficit hyperactivity disorders (ADHD), particularly migraine headache, with caffeine, preferably in combination with another therapeutic agent, such as a beta-blocker.

### BACKGROUND OF THE INVENTION

Headaches have historically been divided into three different classes, based on clinical judgment and symptomatology: tension headache, migraine headache, and cluster headache. Headaches are a common-disorder, with a vast majority of the population suffering from headache at one point during their lives. While there are certainly subjects whose headache classification into one of these three categories may be relatively straightforward, the field has evolved to believe that in point of fact, headache is made up of a spectrum of disease that ranges from tension to migraine, with many variants in between. For instance, one class of headaches is chronic daily headache (CDH), which consists of two main divisions, long-lasting headaches and short-lasting headaches, comprising the following clinical subtypes for each. Long-lasting headaches (i.e., attack duration longer than 4 hours) include transformed migraine (TM), chronic tension-type headache, new daily persistent headache, hemicrania continua, and analgesic round headache. Short-lasting headaches (i.e., attack duration less than 4 hours) include chronic cluster headache, chronic paroxysmal hemicrania, hypnic headache, and idiopathic stabbing headache. Another short-lasting headache type is post-lumbar puncture headache, which is a headache that occurs frequently after patients have undergone lumbar puncture.

Migraine headache (“migraine”), which is often considered the most severe and debilitating in the spectrum of headache categories, is a common disorder, believed to afflict as much as 20 percent of the population, some transiently, some chronically. In migraine patients, throbbing head pain occurs at intervals. The pain often is associated with symptoms such as nausea, vomiting, phonophobia, and photophobia.

The biochemical mechanisms underlying headache in general, and particularly of migraine, are uncertain. The predominant belief expressed in the literature has been that vasodilation of extracranial vessels, and the subsequent neuronal activation of pain fibers surrounding the extracranial vessels, causes migraine and may cause other types of headache, including tension headache, cluster headache, and chronic daily headache. Treatment efforts, therefore, have been aimed at methods of causing vasoconstriction, which includes drugs such as ergot alkaloids and, most recently, 5-HT1 receptor agonists such as sumatriptan. All of these drugs are thought to initially relieve migraine-associated pain by causing vasoconstriction. Unfortunately, this leads to numerous side effects such as chest pain or pressure, flushing, generalized tingling sensations, nausea, vomiting, pain in the legs and arms, asthenia, drowsiness, and dizziness. Acute ergotism is a particularly pernicious side effect of ergot drugs and is characterized by severe central and peripheral vasoconstriction, nausea, vomiting, diarrhea, colic, headache, vertigo, paresthesia, and possibly convulsive seizures. Patients have, on occasion, found total or partial relief for some forms of migraine through the use of non-prescription analgesics. As outlined by Welch (New Engl J. Med. 329: 1476-1483 (1993)), the initial dosages of such analgesics are typically: aspirin, 500-650 mg; acetaminophen, 500 mg; naproxen sodium, 750-825 mg; tolfenamic acid, 200-400 mg; and, ibuprofen 200 mg. After oral dosing, peak plasma concentrations in normal subjects usually occur at about 1 hour for aspirin and acetaminophen, and between 1 and 2 hours for naproxen sodium, tolfenamic acid, and ibuprofen. However, the absorption of these and other agents during a migraine attack has been shown to be impaired, apparently due to gastric stasis.

Despite the fact that multiple classes of therapeutics are being used for the treatment of migraine, there is a clear need for more efficacious treatment of migraine headache. Similarly, there is a need to treat CNS disorders such as migraine headache with a therapeutic modality with minimal side effects.

### BRIEF DESCRIPTION OF DRAWINGS

FIG. 1 illustrates results from responder analysis (time in minutes unless specified).

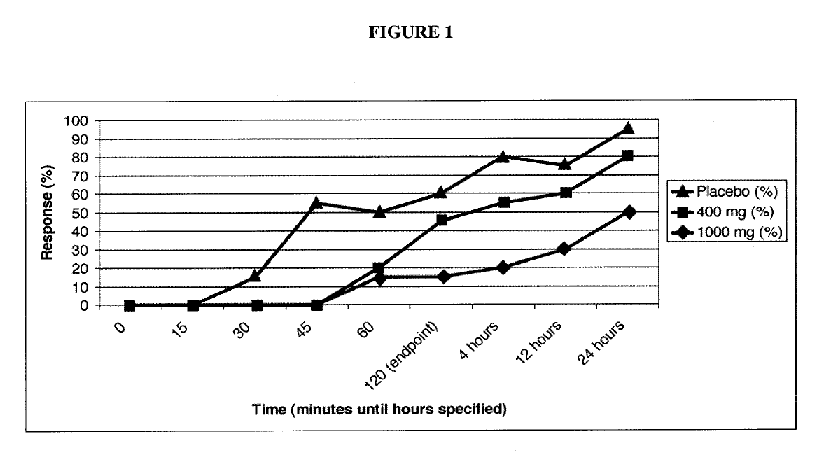

### SUMMARY OF THE INVENTION

The present invention provides compositions, methods and kits for treating or preventing a CNS disorder. In general, the invention involves utilizing caffeine for preventing or alleviating pathological symptoms of a CNS disorder, such as headache, epilepsy, pain, Parkinson's disease, psychiatric disorders such as anxiety, bipolar disorder, depression, and schizophrenia, ADD, and ADHD. In particular, the present invention provides a combination therapy of caffeine and another therapeutic agent, such as a beta-blocker, adrenergic agonist, adrenergic antagonist, calcium channel blocker, antiepileptic medication, tricyclic antidepressant, selective serotonin reuptake inhibitor, methysergide maleate, analgesic, non-steroidal anti-inflammatory drug, serotonin receptor agonist, and ergot derivative.

In some embodiments, caffeine is used in combination with a beta-blocker for the prophylactic or acute treatment of headache, particularly migraine. The method comprises administering to a subject suffering from a headache a pharmaceutical composition comprising caffeine in combination with a beta-blocker, in an amount effective to relieve headache. The headache may be migraine, transformed migraine, chronic daily headache, or post-lumbar puncture headache in mammals, preferably humans.

In various embodiments, compositions and methods of the invention are used to treat headache pain with reduced side effects associated with administering caffeine alone. Furthermore, in some embodiments, the therapeutic effects of caffeine are potentiated and safety profile improved, thus allowing higher doses of caffeine to be used.

In some embodiments, the pharmaceutical compositions of the invention comprise caffeine in the amount ranging from 50 mg to 1.5 gram in combination with another therapeutic agent such as a beta-blocker in the amount ranging from 0.5 mg to 100 mg. (Do we have to limit ourselves to 100 mg? There are beta blockers used in dosages higher than this (nadolol) that we may want to try in the future)

In various embodiments, the pharmaceutical compositions of the invention are administered through a variety of routes of administration, including but not limited to oral, buccal, sublingual, rectal, topical (including skin, mucosal surfaces, airway surfaces), transmucosal, percutaneous, implantable, parenteral (including subcutaneous, intramuscular, intradermal, intravenous and intrathecal), intracranial, intraperitoneal, transdermal, intratracheal, intravaginal, endocervical, intrathecal, intranasal, intravesicular, intraocular, transaural, intravascular, extravascular, intramural, epidural, intraosseous and extramural routes of delivery. In one embodiment, administration is via oral dosing.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In general, the present invention provides compositions and methods of treating a subject who suffers from a CNS disorder utilizing a combination of caffeine and one or more second agent (e.g., one, two, three active ingredients in addition to caffeine). For example, for treating a headache, compositions and methods comprise administering to a subject a pharmaceutical composition comprising an effective amount of caffeine with another therapeutic agent, such as a beta-blocker, to relieve headache.

One aspect of the invention is directed to pharmaceutical compositions and methods of using the same comprising a dosage form comprising caffeine and a beta-blocker. Caffeine has a unique combination of pharmacologic actions that include activation of the sympathetic nervous system and constriction of blood vessels. Although the exact mechanism of action for caffeine is not completely understood, caffeine is thought to act through adenosine receptors, and increases sympathetic tone amongst its pleiotropic activities.

However, in adults, higher doses of caffeine (exceeding 150 mg/dose) have been associated with both centrally and peripherally mediated side effects. For example, the peripherally mediated side effects include tremor, diaphoresis, palpitations, increased blood pressure and heart rate. Furthermore, centrally mediated side effects include anxiety, nervousness, irritability, and sleeplessness. These effects are seen more frequently and increasing severity at higher doses of caffeine. In addition, higher doses of caffeine may increase the rate of cardiac arrhythmias, a potentially life-threatening side effect. These are some of the dose-limiting toxicities that have prevented the clinical utility of caffeine at doses exceeding 150 mg/dose as pharmaceutical compositions.

To potentiate the therapeutic effects of caffeine while avoiding or reducing the side effects of caffeine at higher doses, the present invention provides an innovative combination therapy involving administration of caffeine in combination with another therapeutic agent, such as a beta-blocker, adrenergic agonist, adrenergic antagonist, calcium channel blocker, antiepileptic, tricyclic antidepressant, selective serotonin reuptake inhibitor, methysergide maleate, analgesic and non-steroidal anti-inflammatory, serotonin receptor agonist, and ergot derivative.

In some embodiments, caffeine is used in combination with a beta-blocker for the prophylactic or acute treatment of headache, particularly migraine. It is believed that a combination of caffeine and a beta-blocker may have a significant therapeutic efficacy in preventing the onset of headache and/or alleviate the symptoms of acute headache by synergistically modulating vascular tone of cerebral blood vessels while reducing the side effects of caffeine.

Leveraging their knowledge of the mechanisms of action of CNS disorders, caffeine and beta-blockers, the inventors believe that i) as a class of pharmaceuticals, beta-blockers block activation of the sympathetic nervous system, and thus block the sympathetic stimulatory effects of caffeine, leading to the decrease in heart rate, sweat production, blood pressure, and tremor; and ii) since beta-blockers can enhance caffeine's ability to cause vasoconstriction, and thus combining caffeine plus beta-blockers has a synergistic effect on vasoconstriction of blood vessels. In particular, in the case of the treatment of headache, particularly migraine, the combination of caffeine and a beta-blocker enables a significant therapeutic efficacy with a strong safety profile. The pharmacokinetic and drug interaction profile of the pharmaceutical combination are ideal for treating patients with headaches, particularly migraine, as the inventors believe that beta-blockers can enhance caffeine's vasoconstrictive effects while inhibiting caffeine's broader sympathetic activation and minimizing side effects as a consequence thereof.

A beta-blocker, also called beta-adrenergic blocking agent, is a drug that inhibits the excitatory effects of norepinephrine released from sympathetic nerve endings at beta-adrenergic receptors. In one embodiment the beta-blocker is able to penetrate the blood-brain barrier such that it can potentiate caffeine's action in the central nervous system. Beta-blockers that fit this profile include acebutolol, atenolol, carvedilol, betaxolol, levobunolol, cartelol, metoprolol, nadolol, oxprenolol, pindolol, propranolol, sotalol, and timolol. In addition, propranolol has the benefit of maintaining a high degree of CNS penetration in comparison to other beta blockers. In another embodiment the beta-blocker is a drug that is rapidly absorbed, with a relatively short time to peak plasma levels, such that the activity of the beta-blocker occurs on the same timescale as that of caffeine. Timolol represents an embodiment given its property of rapid absorption and ability to cross the blood-brain barrier.

In some embodiments, caffeine is used in combination with another therapeutic agent for preventing or treating a CNS disorder. Examples of the other therapeutic agent include, but are not limited to, adrenergic agonists, adrenergic antagonists, calcium channel blockers (e.g. verapamil, amlodipine), antiepileptic medications (e.g. valproic acid, topiramate, gabapentin), tricyclic antidepressants (e.g. amitriptyline, nortriptyline, desipramine), selective serotonin reuptake inhibitors (e.g. paroxetine, fluoxetine, sertraline), methysergide maleate, analgesics and non-steroidal anti-inflammatories (e.g. aspirin, ibuprofen, indomethacinacetaminophen), serotonin receptor agonists (e.g. sumatriptan, naratriptan, almotriptan, zolmitriptan), and ergot derivatives (dihydroergotamine, but preferably excluding ergotamine tartrate).

Examples of CNS disorders include, but are not limited to, headache (particularly migraine headache), epilepsy, pain, Parkinson's disease, psychiatric disorders such as anxiety, bipolar disorder, depression, schizophrenia, attention deficit disorders (ADD), and attention deficit hyperactivity disorders (ADHD).

The compounds of the present invention can be applied by any of the accepted modes of systemic administration for agents which affect the central nervous system (CNS) including oral, parenteral, rectal, and otherwise systemic routes of administration. Any pharmaceutically acceptable mode of administration can be used, including solid, semi-solid, or liquid dosage forms, such as, for example, tablets, suppositories, pills, capsules, powders, liquids suspensions, or the like, preferably in unit dosage form suitable to single administration of precise dosages, or preferably in rapid release formulations to maximize time to pharmacologic effect and minimize time to relief of the CNS disorder, particularly migraine headache. The compositions typically include a conventional pharmaceutical carrier or excipient, and the drug product caffeine and another therapeutic agent such as a beta-blocker, and, in addition, can include other medicinal agents, pharmaceutical agents, carriers, etc.

As used herein the terms “compounds”, “compositions”, “pharmaceutical compounds”, “pharmaceutical compositions”, and singular forms thereof are used interchangeably.

The compositions are advantageously and preferably compounded into unit dosage forms containing a predetermined, standard amount of the active compounds (e.g., caffeine and a beta-blocker), to make dosing and patient compliance simpler.

Alternatively, caffeine may be (co)administered with another therapeutic agent to the host in need thereof. (Co)administration within the context of this invention may be taken to mean administration, coadministration, or both. Coadministration in the context of this invention may be defined to mean the administration of more than one therapeutic in the course of a coordinated treatment to achieve an improved clinical outcome. Such coadministration may also be coextensive, that is, occurring during overlapping periods of time, or sequentially, that is, administering caffeine before or after a predetermined period time of administration of the other therapeutic agent.

The amount of active compound administered depends on the subject being treated, the severity of the affliction, the manner of administration, and the judgment of the prescribing physician. In various embodiments, a dosage form disclosed herein comprises a dose of caffeine that is at least 50 mg, 100 mg, 200 mg, 250 mg, 300 mg, 350 mg, 400 mg, 450 mg, 500 mg, 550 mg, 600 mg, 650 mg, 700 mg, 750 mg, 100-2000 mg, 150-1500 mg, 200-1200 mg, 300-1200 mg, 400-1000 mg, 500-800 mg, or 500-600 mg per dose or administration. [&&comment on safety issues for rebuttals]. In another embodiment the dose is 1000 mg, a dose at which side effects can be mitigated by co-administration with a low dose of a beta-blocker.

In various embodiments of a combination dosage formulation of the invention comprises caffeine as described herein, and further comprises a beta-blocker at the low end of what has been pharmacologically effective in treating hypertension, preferably in about 10%, 20%, 30%, 40% or 50% of the amount effective for treating hypertension. For example, in some embodiments, the beta-blocker (e.g., propranolol, timolol) doses are in the range of 10-200 mg, 10-100 mg, 20-100 mg, or 30-60 mg for propranolol and 5-100 mg, 10-80 mg, 10-60 mg, or 20-60 mg for timolol. The dose is preferably administered all at once for acute treatment of headache. The dose is effective in treating all types of headache, but preferably migraine headache. The dosage of these compounds may vary in accordance with the administration route, the age of the patient and the degree of the therapeutic effect desired.

In one embodiment, the pharmaceutical compositions of the invention comprise caffeine and a beta-locker (e.g., propranolol) that is less than about 200 mg, less than about 150 mg, less than about 100 mg, less than about 90 mg, less than about 80 mg, less than about 75 mg, less than about 70 mg, less than about 65 mg, less than about 60 mg, less than about 55 mg, less than about 50 mg, less than about 45 mg, less than about 45 mg, less than about 40 mg, less than about 35 mg, less than about 30 mg.

In some embodiments the doses of the combination of caffeine and propranolol are in a ratio that maximizes the dose of caffeine while minimizing the dose of propranolol. For example, in various embodiment the doses include caffeine doses between about 200 to about 1500 mg caffeine, including but not limited to about 300 mg to about 1200 mg, about 500 mg to about 1300 mg, or 400 mg to about 1000 mg; with doses of propranolol between about 30 to about 60 mg, including but not limited to about 30 mg to about 40 mg, about 40 mg to about 50 mg, about 50 mg to about 80 mg, about 70 mg to about 80 mg, or about 60 mg to about 80 mg. In one embodiment, the caffeine dose is 400 mg and the propranolol dose is 40 mg. In another embodiment, the caffeine dose is 1000 mg and the propranolol dose is 40 mg. The ratio of caffeine to propranolol can be 3 for doses of caffeine between 300-600 mg, and ≧15 for doses of caffeine between 600 mg and 1200 mg. In one embodiment of the pharmaceutical composition, the dose of caffeine is 400 mg and the dose of propranolol is less than 50 mg. In another embodiment of the pharmaceutical composition, the does of caffeine is 1000 mg and the dose of propranolol is 60 mg or less.

In one aspect of the invention, pharmaceutical compositions comprise caffeine and a beta-blocker, and alternatively one or more additional therapeutic agents as described herein. In various embodiments, such compositions are configured to provide mg of caffeine per kg body weight, including but not limited to more than about 5 mg/kg, more than about 6 mg/kg, more than about 7 mg/kg, more than about 8 mg/kg, more than about 9 mg/kg, or more than about 10 mg/kg.

The compounds of the present invention are usually administered in the form of a pharmaceutical composition in an admixture with a pharmaceutical carrier. The pharmaceutical composition can be in the dosage forms such as tablets, capsules, granules, fine granules, pills, lozenges, cachets, dragees, powders, liquids, liquid emulsions, microemulsions, solutions, suspensions, elixirs, syrups, suppositories, injections, or the like. These preparations can be prepared by conventional methods. Compounds of the present invention can be adapted for use in various dosage forms known in the art, such as dosage forms described in Axt et al. U.S. Patent Application Pub. No 2007/0298112 A1 and Hwang U.S. Patent Application Pub. No. 2007/0298105 A1, the disclosure of each of which is herein incorporated by reference.

In various embodiments, pharmaceutical compositions further comprise one or more carriers. Examples of carriers useful include but are not limited to one or more of all organic or inorganic carrier materials that are usually used for the pharmaceutical preparations and are inert to the active ingredient. Examples of the carriers suitable for the preparation of tablets capsules, granules and fine granules include but are not limited to diluents such as lactose, starch, sucrose, D-mannitol, calcium sulfate, or microcrystalline cellulose; disintegrators such as sodium carboxymethylcellulose, modified starch, or calcium carboxymethylcellulose; binders such as methylcellulose, gelatin, acacia, ethylcellulose, hydroxypropylcellulose, or polyvinylpyrrolidone; lubricants such as light anhydrous silicic acid, magnesium stearate, talc, or hydrogenated oil; or the like. When formed into tablets, they may be coated in a conventional manner by using conventional coating agents such as calcium phosphate, carnauba wax, hydroxypropyl methylcellulose, macrogol, hydroxypropyl methylphthalate, cellulose acetate phthalate, titanium dioxide, sorbitan fatty acid ester, or the like.

In various embodiments, pharmaceutical compositions are formulated into a liquid formulation. As such, examples of carriers suitable for the preparation of syrups include but are not limited to sweetening agents such as sucrose, glucose, fructose, or D-sorbitol; suspending agents such as acacia, tragacanth, sodium carboxymethylcellulose, methylcellulose, sodium alginate, microcrystalline cellulose, or veegum; dispersing agents such as sorbitan fatty acid ester, sodium lauryl sulfate, or polysorbate 80; or the like. When formed into syrups, the conventional flavoring agents, aromatic substances, preservatives, or the like may optionally be added thereto. The syrups may be in the form of dry syrup that is dissolved or suspended before use.

In some embodiments, pharmaceutical compositions of the invention are prepared as a suppository. Examples of bases used for the preparation of suppositories include but are not limited to cacao butter, glycerin saturated fatty acid ester, glycerogelatin, macrogol, or the like. When formed into suppositories, the conventional surface active agents, preservatives or the like may optionally be admixed.

In some embodiments, pharmaceutical compositions of the invention are formulated into a liquid formulation (or powder formulation to be reconstituted). When formed into injections, the compound is dissolved in distilled water for injection, to which may optionally be added the conventional solubilizers, buffering or pH adjusting agents, isotonic agents, preservatives and other suitable substances. The injections can be in the solid dry preparations, which are dissolved before use.

These pharmaceutical compositions usually contain caffeine and beta-blocker as the active ingredient in an amount of 0.5% by weight or more, preferably 10 to 70% by weight, based on the total weight of the composition. These compositions may optionally contain other therapeutically active compounds.

In yet other embodiments, pharmaceutical compounds of the invention are formulated into a solid composition. For solid compositions, conventional carriers useful with pharmaceutical compositions of the invention include but are not limited to mannitol, lactose, starch, magnesium stearate, sodium saccharin, talcum, cellulose, glucose, sucrose, magnesium carbonate, and the like may be used. The active compound as defined above may be formulated as suppositories using, for example, polyalkylene glycols, for example, propylene glycol as a carrier. Liquid pharmaceutically administrable compositions can, for example, be prepared by dissolving, dispersing, etc. an active compound as defined above and optional pharmaceutical adjuvants in a carrier, such as, for example, water, saline, aqueous dextrose, glycerol, ethanol, and the like to thereby form a solution or suspension. If desired, the pharmaceutical composition to be administered may also contain minor amounts of non-toxic auxiliary pH buffering agents and the like, for example, sodium acetate, sorbitan monolaurate, triethanolamine oleate, etc. Actual methods of preparing such dosage forms are known, or will be apparent to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount effective to alleviate the symptoms of the subject being treated.

In some embodiments, dosage forms of the invention comprise caffeine and one or more additional pharmaceutical agent (e.g., beta-blocker, such as propranolol), comprising active ingredients in the range of 0.25 to 95% with the balance made up from non-toxic carrier may be prepared. For oral administration, a pharmaceutically acceptable non-toxic composition is formed by the incorporation of any of the normally employed excipients, and may contain 1%-95% active ingredient, preferably 5%-50%. In another aspect of the invention, pharmaceutical compositions comprising one or more excipient, caffeine and a beta-blocker (e.g., propranolol), wherein the one or more excipient enhance absorption of caffeine and/or beta-blocker. These dosage forms may enhance the efficacy of the composition in the treatment of migraine headache. For oral administration, a pharmaceutically acceptable composition may include effervescent excipients.

“Effervescent” means that the dosage form, when mixed with liquid, including water and saliva, will evolve a gas. Preferred effervescent agents (or effervescent couple) evolve gas by means of a chemical reaction which takes place upon exposure of the effervescent disintegration agent to water and/or to saliva in the mouth. This reaction is most often the result of the reaction of a soluble acid source and an alkali monocarbonate or carbonate source. The reaction of these two general compounds produces carbon dioxide gas upon contact with water or saliva. Such water-activated materials must be kept in a generally anhydrous state and with little or no absorbed moisture or in a stable hydrated form, since exposure to water will prematurely disintegrate the tablet. Of course, an effervescent couple (or the individual acid and base separately) can be coated with an LSP coating to prevent premature reaction. Such a couple can also be mixed with previously lyophilized particles coated with an LSP coating as described herein.

Examples of effervescent dosage forms include tablets, granules, and powders. Effervescence can be achieved with a combination of a base and a neutralizing agent such as an acid to form carbon dioxide. Effervescent disintegration agents useful in the present invention can be anything known to be used as an effervescent disintegration, such as described in Wehling et al., U.S. Pat. No. 5,178,878 cols. 5-7, incorporated by reference herein. The acid sources or acid for the effervescent agent may be any which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations of the present invention were intended to be dissolved in a glass of water.

Excipient acids include citric acid, tartaric acid, malic acid, fumaric acid, adipic acids, succinic acids, succinic anhydride, citric anhydride, sodium dihydrogen phosphate, disodium dihydrogen pryophosphate, acid citrate salts, and sodium acid sulfite. Excipient bases that may be used include sodium glycine carbonate, L-lysine carbonate, arginine carbonate, sodium bicarbonate, sodium carbonate, potassium bicarbonate, potassium carbonate, magnesium carbonate, sodium sesquicarbonate, and amorphous calcium carbonate. Other effervescent formulations may also be used that result in the formation of other gases that are safe for human consumption, including oxygen.

The effervescent disintegration agent(s) of the present invention is not always based upon a reaction which forms carbon dioxide. Reactants which evolve oxygen or other gasses which are pediatrically safe are also considered within the scope. Where the effervescent agent includes two mutually reactive components, such as an acid source and a carbonate source, it is preferred that both components react completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. However, in other embodiments of the present invention, the amount of either acid or carbonate source may exceed the amount of the other component. This may be useful to enhance taste and/or performance of a tablet containing an overage of either component. In this case, it is acceptable that the additional amount of either component may remain unreacted.

In general, the amount of effervescent disintegration agent of the present invention useful for the formation of a dosage form according to the present invention should range from about 0% to about 10% of the final composition. Effervescent formulations are known in the art and are described in Lieberman and Lachman, “Pharmaceutical Dosage Forms: Tablets, Volume 1” 1989; Swarbrick and Boylan, “Encyclopedia of Pharmaceutical Technology”, 2000; and U.S. Pat. No. 6,764,696; U.S. Application Publication Nos: 20080031947 and herein incorporated by reference in their entirety. Alternative formulation technologies that enhance oral absorption that are known to one skilled to the art may also be employed in combination with caffeine and a beta-blocker.

The pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL™ 200 (W.R. Grace Co., Baltimore, Md.) and CAB-O-SIL™ (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL™ (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN™ 20), polyoxyethylene sorbitan monooleate 80 (TWEEN™ 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisultite, sodium metabisultite, thiodipropionic acid and its esters, and dithiocarbamates.

The pharmaceutical compositions provided herein for oral administration may be also provided in the forms of liposomes, micelles, microspheres, or nanosystems. Micellar dosage forms can be prepared as described in U.S. Pat. No. 6,350,458.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as other alpha-adrenergic receptor modulators.

Therefore, in various embodiments, any of the pharmaceutical compositions containing caffeine and a beta-blocker described above may include one or more excipients selected from the group consisting of citric acid, tartaric acid, malic acid, fumaric acid, adipic acids, succinic acids, succinic anhydride, citric anhydride, sodium dihydrogen phosphate, disodium dihydrogen pryophosphate, acid citrate salts, and sodium acid sulfite, sodium glycine carbonate, L-lysine carbonate, arginine carbonate, sodium bicarbonate, sodium carbonate, potassium bicarbonate, potassium carbonate, sodium sesquicarbonate, and amorphous calcium carbonate. In one embodiment, pharmaceutical compositions of the invention comprise caffeine and propranolol in dosages described herein and with one or more excipients conventionally used or disclosed herein.

In various embodiments, pharmaceutical compositions of the invention are administered through parenteral administration. Parenteral administration is generally characterized by injection, whether subcutaneously, intramuscularly, or perineurally. Injectables can be prepared in conventional forms, either as liquid solutions, suspensions, or emulsions. Suitable excipients include, for example, water, saline, aqueous dextrose, glycerol, ethanol or the like. In addition, the pharmaceutical compositions may also contain one or more additional substances such as wetting or emulsifying agents, auxiliary pH buffering agents and the like, such as, sodium acetate, sorbitan monolaurate, triethanolamine oleate, etc.

The percentage of active compound contained in such parenteral compositions is highly dependent on the specific nature thereof, as well as on the activity of the compound and the needs of the subject. However, relative to the total amount of ingredients in parenteral compositions, percentages of active ingredient disclosed herein are in amounts of 0.1% to 10%, and preferably 0.2-2%.

Parenteral administration is through any convention means, such as by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to known methods (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including .alpha.-cyclodextrin, .beta.-cyclodextrin, hydroxypropyl-.beta.-cyclodextrin, sulfobutylether-.beta.-cyclodextrin, and sulfobutylether 7-.beta.-cyclodextrin (CAPTISOL™, CyDex, Lenexa, Kans.).

The pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer

The pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol™; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The pharmaceutical compositions provided herein may be micronized to a size suitable for delivery by inhalation, such as about 50 micrometers or less, or about 10 micrometers or less. Particles of such sizes may be prepared using a comminuting method, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

The pharmaceutical compositions provided herein for topical administration may be formulated to be immediate release or modified release, including delayed-, sustained-, pulsed-, controlled-, targeted, and programmed release.

The pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

In some embodiments, pharmaceutical compositions of the invention are formulated into formulations configured to allow immediate release of one or more active ingredients. For example, in some embodiments, a dosage form is configured for immediate release of caffeine, immediate release of a beta-blocker (e.g., propranolol) or immediate release of caffeine and propranolol. For immediate release, the pharmaceutical compositions may be formulated in a pharmaceutical composition that enables fast release, including but not limited to rapidly dissolving tablets, films, and chewable tablets according to methods known in the art. Various buffers and pH modulation of the formulation using acids and/or bases generally available in the pharmaceutical field and may be used to improve the solubility of caffeine and/or the beta-blocker, as well as enable absorption of either or both pharmaceuticals across the buccal mucosa for a rapidly acting formulation.

In various embodiments, for example, caffeine and the other therapeutic agent may be orally administered as a multi-layered tablet. In some embodiments, the multilayered tablet may be a bilayered tablet, wherein the bilayered tablet is comprised of an inner core layer surrounded by an outer layer.

In one embodiment, a multi-layered tablet or pill is configured to comprise an immediate release layer and a delayed or sustained layer. Such multi-layered oral dosage forms are composed using conventional technologies. In one embodiment, a dosage form comprises an immediate-release layer comprising caffeine and a sustained or delayed release layer comprising propranolol, caffeine and propranolol. In another embodiment, the immediate release layer comprises propranolol, caffeine and propranolol, or caffeine, propranolol and another active ingredient disclosed herein (e.g., additional beta-blocker).

In some embodiments, the bilayered tablet can be comprised of an outer layer containing one dose of caffeine and one or more excipients and an inner layer comprised of one dose of a beta blocker and one or more expicients. In one embodiment, the beta blocker is propranolol.

In some embodiments, the bilayered tablet can be comprised of an outer layer containing one dose of a beta blocker and one or more excipients and an inner layer comprised of one dose of caffeine and one or more excipients. In one embodiment, the beta-blocker is propranolol.

In some embodiments, the bilayered tablet can be comprised of an outer layer containing one dose of caffeine, one dose of a beta blocker, and 1 or more excipients, and an inner layer containing one dose of caffeine, one dose of a beta blocker, and 1 or more excipients, wherein the doses of caffeine and beta blocker in the inner layer are both higher than the doses of caffeine and beta blocker in the outer layer, respectively. In one such embodiment, the beta blocker is propranolol.

In some embodiments, the outer layer of a bilayered tablet is configured for immediate release, thus providing plasma levels of active agents in about 1 minute to 15 minutes, about 5 minutes to about 45 minutes, about 5 minutes to about 15 minutes, about 1 minute to about 30 minutes; and the inner layer is configured to provide plasma levels of the active ingredients therein in about 30 minutes to 4 hours, about 10 minutes to 1, 2, 3, 4, 5, 6, 7 or 8 hours, from about 30 minutes to several hours (e.g., 4, 5, 6, 7, 8, 9, 10, 11, 12 hours). In any of the embodiments disclosed herein, the immediate release layer comprise one, two or three active agents and the sustained release layer comprise one, two or three active agents. For example, the immediate release layer comprises caffeine, caffeine-propranolol, caffeine-propranolol and a third active agent; and the sustained release layer comprises caffeine, caffeine-propranolol, caffeine-propranolol and a third active agent. Therefore, in one embodiment, a bilayered tablet comprises at least two, three, four, five or six pharmaceutical agents in a therapeutically effective amount.

The bilayered tablet can be a variety of sizes and shapes, and examples of shapes of the bilayered tablet include, but are not limited to, oblong, oval, spherical, rod, or block. The size and shape of the tablet may impact the release rate of each layer. Pharmaceutical compositions of the invention can be manufactured using conventional methods, such as those disclosed in Benkerrour et al. U.S. Patent Application Pub. No. 2004/0115265 A1, Eisenreich U.S. Patent Application Pub. No. 2006/0110450 A1, Lewis et al. U.S. Patent Application Pub. No. 2007/0275054 A1, and Deboeck et al. U.S. Patent Application Pub. No. US2007/0160663 A1, which are herein incorporated by reference.

Other modes of administration can also be practiced in accordance with the present invention. For example, buccal, sublingual, rectal, topical (including skin, mucosal surfaces, airway surfaces), percutaneous, implantable, parenteral (including subcutaneous, intramuscular, intradermal, intravenous and intrathecal), intracranial, intraperitoneal, transdermal, intratracheal, intravaginal, endocervical, intrathecal, intranasal, intravesicular, intraocular, transaural, intravascular, extravascular, intramural, epidural, intraosseous and extramural routes of delivery are examples of delivery methods that are contemplated by the present invention.

In some embodiments, caffeine and the other therapeutic agent may be administered separately or in a unit dosage form transmucosally to the host in need thereof to allow fast introduction of the potent, fast-acting drugs into the bloodstream, and/or in a dose-to-effect manner such that sufficient drug is administered to produce precisely the desired effect. Via transmucosal administration, the drugs may be introduced into the patient's bloodstream almost as quickly as through injection, and much more rapidly than using the oral administration route, while avoiding the negative aspects of both methods. In one embodiment, the other therapeutic agent is a beta blocker, such as propranolol.

For transmucosal administration, caffeine and the other therapeutic agent can be jointly or separately compounded into a dissolvable matrix material. The dissolvable matrix may include carbohydrates, fats, proteins, waxes (natural and synthetic), hydrocarbons, and other materials which safely dissolve in the mouth. The dissolvable matrix, or dosage-form, can be used to administer drugs in a dose-to-effect manner, or until the precise desired effect is achieved. The dosage-form preferably has an appliance or handle attached thereto to permit removal from the patient's mouth. In one embodiment, the other therapeutic agent is a beta blocker, such as propranolol.

After mixing, the mixture may be compressed, poured into a mold cavity, dehydrated, freeze dried, or otherwise formed as an integral drug delivery system. In some embodiments within the scope of the present invention, specific confectionery components are combined in order for the mixture to form an integral solid mass. These components may include, for example, compressible confectioner's sugar, sorbitol, mannitol, and maltodextrin.

Caffeine and the other therapeutic agent may be incorporated into a flavored dissolvable matrix material and the matrix mixture attached onto an appliance or holder. In use, the present invention provides for the administration of drugs through the mucosal tissue of the mouth, pharynx, and esophagus, thereby avoiding the problems of both injection and oral administration. In one embodiment, the other therapeutic agent is a beta blocker, such as propranolol.

In various embodiments, dosage forms of the invention comprising caffeine, a beta blocker and alternatively one or more additional therapeutic agent are administered in a modified released dosage form. The pharmaceutical compositions provided herein in a modified release dosage form may be fabricated using a matrix controlled release device (see, Takada et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT™, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients in the compositions.

The pharmaceutical compositions provided herein in a modified release dosage form may be prepared by methods, including direct compression, dry or wet granulation followed by compression, melt-granulation followed by compression.

In various embodiments, dosage forms of the invention comprising caffeine, a beta blocker and alternatively one or more additional therapeutic agent are administered with buffering agents. Buffering agents and other types of pH control can also be added simultaneously in order to provide for maximum drug efficiency. It will be appreciated that drugs in the unionized form are more readily transported across the mucosal membrane. Therefore, if pH conditions can be adjusted to maximize the percentage of unionized drug available, the effectiveness of the drug is maximized.

Buffering agents are particularly important for those drugs that partially ionize within the pH range of the mouth, such as weak acid and weak base drugs. Generally, buffering agents are more important when hydrophilic drugs are used because those drugs usually have lower mucosal permeability and dissolve more readily in saliva within the mouth.

Permeation enhancers may also be incorporated within the dissolvable matrix to improve the permeability of the mucosal membrane. The permeability of both lipophilic and nonlipophilic drugs may be improved by using suitable permeation enhancers. Typical permeation enhancers may include bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate. Other permeation enhancers such as sodium dodecyl sulfate (“SDS”), dimethyl sulfoxide (“DMSO”), sodium lauryl sulfate, salts and other derivatives of saturated and unsaturated fatty acids, surfactants, bile salt analogs, derivatives of bile salts, or such synthetic permeation enhancer.

In some embodiments, caffeine and the other therapeutic agent may be administered jointly or separately by using a controlled release dosage form. Controlled release within the scope of this invention can be taken to mean any one of a number of extended release dosage forms. Extended release dosage forms are described in Heaton et al. U.S. Patent Application Pub. No. US2005/0129762 A1 and Edgren et al. U.S. Patent Application Pub. No. 2007/0128279 A1, which are herein incorporated by reference. Time-release formulations are known in the art and are described in Sawada et al. U.S. Patent Application Pub. No. 2006/0292221 A1, which is herein incorporated by reference.

The following terms may be considered to be substantially equivalent to controlled release for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to, physical systems and chemical systems.

In various embodiments, caffeine and one or more additional therapeutic agent (e.g., beta blocker, such as propranolol) are configured for delivery using physical systems. Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., non-erodible, erodible, environmental agent ingression, and degradable), and materials physically dispersed in non-porous, polymeric, or elastomeric matrices (e.g., non-erodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

The method of the present invention can be used with other therapeutic agents commonly used to treat headache acutely, thus enhancing the effects of therapeutic agents and adjunctive agents.

In another aspect of the invention, compound of the invention comprising caffeine and one or more additional therapeutic agents as described herein are administered in a therapeutically effective amount to an animal, especially a human, for treating or preventing a CNS disorder. In various embodiments, pharmaceutical compositions are administered to a subject to treat or prevent a CNS disorder including but not limited to migraine headaches (with or without aura), cluster headaches, chronic headaches, tension type headaches, Hemicrania Continua, new daily persistent headaches, chronic tension type headaches or any combination thereof. In yet other embodiments, pharmaceutical compositions are administered to a subject to treat or prevent a CNS disorder including but not limited to epilepsy, pain, Parkinson's disease, psychiatric disorders such as anxiety, bipolar disorder, depression, and schizophrenia, ADD, and ADHD.

As used herein, “therapeutically effective amount” shall mean the dosage of drug that provides the specific pharmacological response for which the drug is administered in a significant number of subjects in need of such treatment. In particular, it is emphasized that migraine headache is not well understood, and the etiology of particular migraines may vary, as does the response to particular drugs. Thus reference to “specific pharmacological response for which the drug is administered in a significant number of subjects in need of such treatment” is a recognition that a “therapeutically effective amount,” administered to a particular subject in a particular instance will not always abort a migraine attack or relieve an actual migraine headache, even though such dosage is deemed “therapeutically effective” by those skilled in the art. It is to be further understood that drug dosages are, in particular instances, measured as oral dosages, or parenteral or inhaled dosages or with reference to drug levels as measured in blood.

The rate of achieving a therapeutic effect of a dosage in a patient can be evaluated using a scale for assessing pain. One scale that can be used to measure pain levels is a Likert scale. The pain levels in the Likert scale can be defined, such as severe, moderate, mild, or none. Different dosages can cause pain to be perceived at different levels. The dosage may decrease pain to different levels at different rates. The rate at which a dosage provides a therapeutic effect can be 120 minutes, more than 120 minutes, or less than 120 minutes.

Optionally, high doses are sometimes required for some therapeutic agents to achieve levels to effectuate the target response, but high doses often associate with a greater frequency of dose-related adverse effects. Thus, combined use of the pharmaceutical composition of the present invention with therapeutic agents commonly used to treat headache allows the use of relatively lower doses of other agents, which results in a lower frequency of adverse side effects associated with long-term administration of such agents. Thus, another advantage of the compounds in this invention is to reduce adverse side effects of drugs used to treat headache, such as tolerance, dependence, constipation, respiratory depression, sedation, and gastrointestinal side effects.

According to the present invention, examples of the other therapeutic agent include, but are not limited to, calcium channel blockers (e.g. verapamil, amlodipine), antiepileptic medications (e.g. valproic acid, topiramate, gabapentin), tricyclic antidepressants (e.g. amitriptyline, nortriptyline, desipramine), selective serotonin reuptake inhibitors (e.g. paroxetine, fluoxetine, sertraline), methysergide maleate, analgesics and non-steroidal anti-inflammatory drugs (NSAIDs, e.g. aspirin, acetaminophen; ibuprofen; flurbiprofen; ketoprofen; naproxen; oxaprozin; etodolac; indomethacin; ketorolac; nabumetane; piroxicam; celecoxib; rofecoxib; meloxicam; JTE-522; L-745,337; and NS398), serotonin receptor agonists (e.g. sumatriptan, naratriptan, almotriptan, zolmitriptan, frovatriptan, nzatriptan), and ergot derivatives (dihydroergotamine, ergotamine tartrate).

Caffeine may also be combined with metoclopramide, a drug known to relieve migraine-associated nausea when administered at a minimum oral dose of 10 mg. Metoclopramide hydrochloride monohydrate is conveniently provided in conventional tablets of 5 and 10 mg, as a solution of 5 mg/5 ml and as an injectable solution of 5 mg/ml.

Dosages of analgesics will be adjusted by physicians based upon clinical factors. Nevertheless, some generalizations can be made. Indomethacin should be useful when present in tablets in a range of from about 25 to 75 mg, when present in suppositories at about 50 mg, and when in oral suspensions at a concentration of about 25 mg/5 ml. A typical daily oral dosage of indomethacin is three 25 mg doses taken at intervals during one day. However, daily doses of up to about 150 mg are also useful in some subjects. Sustained release dosage forms of indomethacin are also available and provide longer lasting blood levels than conventional tablets. In particular, a 25 mg sustained release dosage form can be used as an alternative to 25 mg three times daily or 75 mg twice daily can be substituted for 50 mg three times daily.

Ibuprofen is conveniently provided in tablets or caplets of 50, 100, 200, 300, 400, 600, and 800 mg and as a suspension of 100 mg/5 ml. Daily doses should not exceed 3200 mg and doses should be individualized. 200 mg-800 mg may be particularly useful when given 3 or 4 times daily.

Flurbiprofen is useful when in tablets at about 50 to 100 mg. Daily doses of about 100 to 500 mg, and particularly about 200 to 300 mg total are usually effective.

Ketoprofen is particularly useful when contained in capsules in an amount of about 25 to 75 mg. Daily doses of about 100 to 500 mg, and particularly about 100 to 300 mg are useful, as is about 25 to 50 mg every six to eight hours.

Naproxen is particularly useful when contained in tablets of from 250 to 500 mg, and in oral suspensions of about 125 mg/5 ml. For naproxen sodium, tablets of about 275 or about 550 mg are particularly useful. Initial doses of about 100 to 1250 mg, and particularly 350 to 800 mg are useful. Another useful dose is about 550 mg.

Oxaprozin is notable for having a pharmacokinetics half-life of 42-50 hours and a bioavailability of 95%. It is usefully provided as caplets of 600 mg. Daily doses of 1200 mg have been found to be particularly useful and daily doses should not exceed 1800 mg or 26 mg/kg. The lowest effective dose should always be used.

Etodolac is usefully provided in capsules of 200 mg and 300 mg and in tablets of 400 mg. Useful doses for acute pain are 200-400 mg every 6-8 hours, not to exceed 1200 mg/day. Patients weighing less than 60 kg are advised not to exceed doses of 20 mg/kg. Doses for other uses are also limited to 1200 mg per day in divided doses, particularly 2, 3, or 4 times daily.

Ketorolac is usefully provided in tablets of 10 mg and as a sterile parenteral preparation for injection in 15 mg/ml and 30 mg/ml dosage forms. Oral doses of up to 40 mg, and particularly 10-30 mg per day and parenteral doses up to 120-150 mg per day have been useful in the amelioration of pain.

Nabumetane may be provided in tablets of between 500 mg and 750 mg. Daily doses of 1500-2000 mg/day after an initial dose of 1000 mg are of particular use.

Mefenamic acid is particularly useful when contained in capsules of about 250 mg. For acute pain such as migraine, an initial dosage of 1 to 1000 mg, and particularly about 500 mg, is useful, though other dosages may be required for specific subjects.

Meclofenamate sodium is provided as capsules of 50 mg and 100 mg. Daily doses of up to 400 mg may be used. Typically a patient will take 50-100 mg every 4-6 hours.

One particular group of long acting NSAIDs that may be used are the cyclooxygenase-2 (“COX-2”) inhibitors, for example: celecoxib, rofecoxib, meloxicam, piroxicam, JTE-522, L-745,337, or NS398, or pharmaceutically acceptable salts thereof. JTE-522, L-745,337 and NS398 are described, inter alia, in Wakitani, et al., Jpn. J. Pharmacol. 78:365-371 (1998); and Panara, et al., Br. J. Pharmacol. 116:2429-2434 (1995). The amount present in a tablet or administered to a patient will depend upon the particular COX-2 inhibitor being used. For example, piroxicam may be present at 10 to 20 mg per tablet. Celecoxib may be administered to a human in an amount of from about 100 mg to about 500 mg or preferably, in an amount of from about 100 mg to about 200 mg.

The present invention also provides a kit or assembly of kits containing caffeine and another therapeutic agent. The kit may contain caffeine and the other therapeutic agent in a uniform dosage in a vessel, or separately in different vessels. The kit may further comprise instruction as to how to use the kit for treating or preventing a disease or condition, for example, a CNS disorder such as migraine headache. The instruction may be in a printed form. The kit may also further contain diluent for the drugs, a syringe for injection, or other means for administering the drugs to the host. Caffeine and another therapeutic agent may be contained in separate kits which are assembled into an assembly of kits for use.

The present invention further provides a business method for commercializing caffeine for treating or preventing a CNS disorder such as migraine headache. The business method comprises: promoting the health benefit of caffeine in the treatment or prevention of a CNS disorder; and selling caffeine to a distributor or a user in need thereof. The step of promoting the health benefit of caffeine may include promoting the health benefit of caffeine in combination with another therapeutic agent, such as a beta-blocker (acebutolol, atenolol, carvedilol, metoprolol, nadolol, oxprenolol, pindolol, propranolol, sotalol, and timolol), calcium channel blockers (e.g. verapamil, amlodipine), antiepileptic medications (e.g. valproic acid, topiramate, gabapentin), tricyclic antidepressants (e.g. amitriptyline, nortriptyline, desipramine), selective serotonin reuptake inhibitors (e.g. paroxetine, fluoxetine, sertraline), methysergide maleate, analgesics and non-steroidal anti-inflammatory drugs (NSAIDs, e.g. aspirin, acetaminophen; ibuprofen; flurbiprofen; ketoprofen; naproxen; oxaprozin; etodolac; indomethacin; ketorolac; nabumetane; piroxicam; celecoxib; rofecoxib; meloxicam; JTE-522; L-745,337; and NS398), serotonin receptor agonists (e.g. sumatriptan, naratriptan, almotriptan, zolmitriptan, frovatriptan, rizatriptan), and ergot derivatives (dihydroergotamine, ergotamine tartrate).

According to the business method, caffeine may be in a form of solid (e.g., powder) or liquid (e.g., dissolved in aqueous solution such as coffee). Caffeine and the other therapeutic agent may be sold as a combination in a unit dosage form, or separately in a separate dosage form. The promotion of the health benefit of caffeine in the treatment or prevention of a CNS disorder may appear in a printed publication, in audio or video media, or on a website over the Internet.

The following examples further illustrate the present invention. These examples are intended merely to be illustrative of the present invention and are not to be construed as being limiting.

### EXAMPLES

### Example I

### Treatment of Migraine with Caffeine and Propranolol

In this example, human subjects suffering from migraine are treated with a combination of caffeine and a beta-blocker, propranolol, according to the present invention.

Subjects with diagnosis of migraine with or without aura as defined by the International Headache Society (1HS) criteria are enrolled. All enrolled subjects are treated for one moderate or severe migraine headache within 60 days of enrollment. Up to 60 subjects are enrolled at up to 15 clinical sites to obtain data on 60 assessable subjects.

Subjects who meet all the inclusion criteria and none of the exclusion criteria provided below are enrolled.

Inclusion Criteria:

1. Subject has a minimum 12-month migraine history that the investigator determines meets the IHS Migraine Diagnostic Criteria for migraine with or without aura.

2. Subject is between 18-50 years of age.

3. Subject experiences an average of 2-8 migraines per month.

4. If on preventive migraine therapy, medication regimen has been stable for 30 days and will remain stable for the duration of participation.

5. Subject is able to communicate adequately and comply with the requirements of the study as determined by the investigator.

6. Subject is able to read and understand the informed consent written in English and voluntarily consents to sign the informed consent form.

Exclusion Criteria:

1. Subject's age of migraine onset is greater than 50 years.

2. Subject has less than 48 hours of freedom from headache between attacks of migraine.

3. Subject meets the criteria for complicated and/or brainstem migraines.

4. Subject is pregnant or lactating.

5. Subject has history of alcohol or drug abuse within the past 2 years.

6. Subject has existing systolic blood pressure <100 mm Hg, existing systolic blood pressure >150 mm Hg, and or heart rate <50 beats per minute.

7. Subject has heart block greater than 1st degree without a functioning pacemaker

8. Subject has a history of tachyarrythmias

9. Subject has uncompensated CHF

10. Subject has severe chronic obstructive pulmonary disease or severe asthma.

11. Subject has consumed caffeine within 6 hours.

12. Subjects with existing generalized anxiety disorder (GAD) and/or panic disorder.

13. Subjects with existing severe hepatic and/or renal insufficiency.

14. Subjects with existing Raynaud's disease.

15. Subject is participating in another clinical trial during or within 30 days prior to study enrollment.

The subjects are randomly assigned to either active or placebo treatment groups in 3 arms. Subjects receiving active treatment receive one of two dosing arms, caffeine/propranolol (mg) 400/40 or 1000/40 via oral administration. Both caffeine and propranolol are in the form of tablets.

Pain levels are recorded in a provided headache diary, using a 0-3 severity rating (“0”=none; “1”=mild; “2”=moderate; “3”=severe) along with the presence or absence of secondary symptoms (nausea, photophobia, and phonophobia). Subjects are instructed to record the pain severity ratings and secondary symptoms in the headache diary at the recommended intervals (baseline prior to dosing; 15, 30, 45, 60 and 120 minutes; 4, 12, and 24 hours after dosing). All time points are calculated from the recorded time of first oral medication administration.

The primary efficacy outcome measure is pain relief at 2 hrs (defined as a decrease in headache pain intensity from severe or moderate headache pain at baseline to moderate or mild respectively at 2 hrs). The secondary efficacy outcome measures are: i) pain-free at 2 hrs post first administration of caffeine/propranolol; and ii) relief of associated symptoms such as nausea, and photophobia and phonophobia at 2 hours.

The efficacy is assessed by analyzing data collected from subject diaries. Headache pain, nausea, photophobia and phonophobia are measured at baseline, 15, 30, 45, 60, and 120 minutes after administration of the first dose. In order to evaluate sustained pain relief, subjects continue to report headache symptoms through 24 hours at the following time points: 4, 12, and 24 hours.

### Example II

### Treatment of Refractory Migraine with Caffeine and Propranolol

Patients from an active headache clinic population are selected for open-label treatment with a combination of caffeine and propranolol according to the present invention. The patients have refractory migraines as defined by International Headache Society criteria. All have failed or responded poorly to trials with other drugs as acute therapy. The combination therapy of caffeine and propranolol is initiated as acute therapy once the patient has experienced a moderate or severe migraine. Patients receive a combination of caffeine/propranolol (mg) 500/40 or 1000/40 via oral administration. Both caffeine and propranolol are in the form of tablets.

Pain levels are recorded in a provided headache diary, using a 0-3 severity rating (“0”=none; “1”=mild; “2”=moderate; “3”=severe) along with the presence or absence of secondary symptoms (nausea, photophobia, and phonophobia). Subjects are instructed to record the pain severity ratings and secondary symptoms in the headache diary at the recommended intervals (baseline prior to dosing; 15, 30, 45, 60 and 120 minutes; 4, 12, and 24 hours after dosing). All time points are calculated from the recorded time of first oral medication administration.

The primary efficacy outcome measures is pain relief at 2 hrs (defined as a decrease in headache pain intensity from severe or moderate headache pain at baseline to moderate or mild respectively at 2 hrs). The secondary efficacy outcome measures are: i) pain-free at 2 hrs post first administration of caffeine/propranolol; and ii) relief of associated symptoms such as nausea, and photophobia and phonophobia at 2 hours.

The efficacy is assessed by analyzing data collected from subject diaries. Headache pain, nausea, photophobia and phonophobia are measured at baseline, 15, 30, 45, 60, and 120 minutes after administration of the first dose. In order to evaluate sustained pain relief, subjects continue to report headache symptoms through 24 hours at the following time points: 4, 12, and 24 hours.

### Example III

### Treatment of Refractory Migraine with Caffeine and Propranolol

A double-blinded, placebo-controlled clinical trial in patients with migraine headache was conducted. Enrolled patients were required to have 2-8 migraines per month and a minimum of a 12-month history of migraines consistent with the International Headache Society (IHS) Migraine Diagnostic Criteria. The trial had 60 patients treated in total, separated into three arms with 20 patients per arm:

1) Placebo [calcium]

2) 400 mg caffeine/40 mg propranolol

3) 1000 mg caffeine/40 mg propranolol

After enrollment, patients were given study medication and were asked to treat the first moderate to severe migraine they suffered and to report the severity of their headache and associated symptoms at baseline and 15, 30, 45, 60, and 120 minutes after dosing. The primary endpoint was the percentage of patients who achieved an improvement in headache severity to a condition of “mild” or “none”. Secondary endpoints included a decrease in the headache pain intensity to “none” at 2 hours post-treatment, safety and tolerability of study drug, need for rescue medication, and subject treatment satisfaction.

Upon completion of the study, no patients dropped out. On an intention to treat basis, the percentage of patients who met the primary endpoint were 20% in the placebo group, 45% in the 400 mg caffeine/40 mg propranolol group, and 60% in the 1000 mg caffeine/40 mg propranolol group. For the 400 mg arm, the Chi-square value was 7.8 with one degree of freedom, two-tailed P=0.005 compared to placebo. For the 1000 mg arm, the Chi-square value was 20, P<0.0001.

Using the Likert scale (0-10) secondary endpoint reaching 0 pain at the 120 minute point, 5% of placebo met the endpoint, 15% of the 400 mg arm met the endpoint, and 35% of the 1000 mg arm met the endpoint. For the 400 mg arm, the Chi-square was 4.2, P=0.04. For the 1000 mg arm, the Chi-square value was 19.2, P<0.0001.

There were no serious adverse events, and only 9 patients had side effects, which ranged from nausea, insomnia, jitteriness, and anxiety. The placebo group (6 subjects) had 2 subjects with nausea, 1 with nausea and head tingling, 1 with nausea and abdominal pain, 1 with dizziness and anxiety, 1 with blurred vision; the 400 mg group (4 subjects) has 2 with nausea, 1 with jittery feeling, 1 with flushed face sensation; and the 1000 mg group (8 subjects) had 2 with nausea and tingling, 2 felt wired, 1 felt restless/anxious, 1 felt nausea and anxiety, 1 had nausea and insomnia, and 1 felt flushed.

Given the magnitude of the caffeine dose that was used in the study and the number of patients receiving high doses of caffeine (40), this is a low incidence of side effects, which is attributable to the combined dosing with propranolol.

Improvement in the secondary endpoint of subject treatment satisfaction on the 11 point (0-10) Likert scale solidly reached statistical significance. 5% of placebo met the endpoint, 40% of the 400 mg arm met the endpoint, and 80% of the 1000 mg arm achieved the endpoint. For the 400 mg arm, the Chi-square was 7.03, P=0.008. For the 1000 mg arm, the Chi-square was 23.0, P=0.0001.

There was also a significantly lower requirement for rescue medications needed in both treatment arms compared to placebo. In the placebo arm, 30% of subjects did not need rescue medications, whereas 65% and 75% of the 400 mg group (Chi-square=4.912, P=0.0267) and 1000 mg group (Chi-square=8.120, P=0.0044), respectively, did not need rescue medications>

Furthermore, the following data was obtained for patients with sustained pain relief from 2-24 hours without the use of rescue meds (ITT): Placebo=33% of pts achieving endpoint required rescue meds; 400 mg=11% pts achieving endpoint required rescue meds; and 1000 mg=8% pts achieving endpoint required rescue meds. This demonstrates that more patients taking the higher dose of caffeine had sustained pain relief, without the need for rescue medications, than those taking the lower dose caffeine or placebo.

HA recurrence—return of HA within 24 hours among those patients with relief after 2 hours of dosing (ITT): Placebo=33% HA recurrence; 400 mg=0% HA recurrence; and 1000 mg=8% HA recurrence. This demonstrates that patients taking either dose of caffeine who achieved pain relief were less likely to have a headache recurrence than those subjects taking placebo.

### Example IV

### Bilayered Tablet with Caffeine Outer Layer and Inner Layer of Propranolol

The pharmaceutical composition can be administered in the form of a bilayered tablet. The outer layer of the tablet comprises caffeine and propranolol. For example, in some embodiments, the caffeine is in a dose of 300 mg, 350 mg, 400 mg, 450 mg, 500 mg, 550 mg, 600 mg, 650 mg, 700 mg, 750 mg, 800 mg, 850 mg, 900 mg, 950 mg or 1000 mg; and the propranolol is in a dose of 30 mg, 40 mg, 50 mg, 60 mg, 70 mg, 80 mg, 90 mg, 100 mg, 110 mg, 115 mg, 120 mg, 125 mg, 130 mg, 135 mg, 140 mg, 145 mg or 150 mg. The outer layer can also contain excipients, including 60 mg lactose, 30 mg mannitol, 5 mg stearic acid, and 5 mg magnesium stearate. The outer layer completely surrounds the inner layer, which forms a core.

The core inner layer of the tablet also comprises caffeine and propranolol, which are in a dose of 300 mg, 350 mg, 400 mg, 450 mg, 500 mg, 550 mg, 600 mg, 650 mg, 700 mg, 750 mg, 800 mg, 850 mg, 900 mg, 950 mg or 1000 mg; and the propranolol is in a dose of 30 mg, 40 mg, 50 mg, 60 mg, 70 mg, 80 mg, 90 mg, 100 mg, 110 mg, 115 mg, 120 mg, 125 mg, 130 mg, 135 mg, 140 mg, 145 mg or 150 mg, respectively.

In various embodiments, the total dosage for a therapeutic agent can be apportioned in the outer/inner layer so that a total dose for caffeine is about: 300 mg, 350 mg, 400 mg, 450 mg, 500 mg, 550 mg, 600 mg, 650 mg, 700 mg, 750 mg, 800 mg, 850 mg, 900 mg, 950 mg or 1000; and the total dose for propranolol is 30 mg, 40 mg, 50 mg, 60 mg, 70 mg, 80 mg, 90 mg, 100 mg, 110 mg, 115 mg, 120 mg, 125 mg, 130 mg, 135 mg, 140 mg, 145 mg or 150 mg; and further where the apportioned percentage of caffeine and propranolol in the outer/inner layer is about 10%/90%, 20%/80%, 30%/70%, 40%/60%, 50%/50%, 60%/40%, 70%/30%, 80%/20%, or 90%/10%, respectively. For example, in one embodiment, a bilayered tablet comprises 500 mg caffeine in the outer layer and 500 mg caffeine in the inner layer (50%/50%) for a total of dosage 1000 mg, while for propranolol, the outer layer comprises 32 mg and the inner layer comprises 48 mg (40%/60%) for a total dosage of 80 mg. This is but one example of the combinations that are disclosed herein above. In various embodiments, the apportionment can be the same for both caffeine and propranolol, or different for each of caffeine and propranolol.

Furthermore, in additional embodiment, the inner layer or outer layer can comprise one or more additional therapeutic agents as described above

The inner layer also contains excipients, including 5 mg microcrystalline cellulose, 20 mg mannitol, and 0.5 mg magnesium stearate. In further embodiments, the bilayered tablet is covered in carnauba wax and is in an oblong shape to facilitate swallowing.

In another embodiment, the outer layer and/or the inner layer comprises an additional therapeutic agent, including but not limited to a beta-blocker, adrenergic agonist, adrenergic antagonist, calcium channel blocker, antiepileptic medication, tricyclic antidepressant, selective serotonin reuptake inhibitor, methysergide maleate, analgesic, non-steroidal anti-inflammatory drug (NSAID), serotonin receptor agonist, and ergot derivative, which are disclosed herein, or known.

TABLE 1
Caffeine Responder Analysis

| | Time (minutes | | | | | | | | |
|---|---|---|---|---|---|---|---|---|---|
| | unless specified) | | | | | | | | |
| | | | | | | 120 | | 12 | 24 |
| | 0 | 15 | 30 | 45 | 60 | (endpoint) | 4 hours | hours | hours |
| Placebo (%) | 0 | 0 | 0 | 0 | 15 | 15 | 20 | 30 | 50 |
| 400 mg (%) | 0 | 0 | 0 | 0 | 20 | 45 | 55 | 60 | 80 |
| 1000 mg (%) | 0 | 0 | 15 | 55 | 50 | 60 | 80 | 75 | 95 |

As Table 1 illustrates in one study two different dosages of caffeine achieved statistically significant improvements in comparison to placebo as well as between the two dosages of caffeine. The endpoint in this trial was measurement of pain using conventional criteria.

The invention, and the manner and process of making and using it, are now described in such full, clear, concise and exact terms as to enable any person skilled in the art to which it pertains, to make and use the same. It is to be understood that the foregoing describes embodiments of the present invention and that modifications may be made therein without departing from the scope of the present invention as set forth in the claims. To particularly point out and distinctly claim the subject matter regarded as invention, the following claims conclude the specification.

What is claimed is:

1. A method of treating a central nervous system (CNS) disorder in a subject in need of such treatment, comprising: administering to the subject a therapeutically effective amount of caffeine in combination with another non-caffeine therapeutic agent.

2. The method according to claim 1, wherein the CNS disorder is selected from the group consisting of headache, epilepsy, pain, Parkinson's disease, psychiatric disorders such as anxiety, bipolar disorder, depression, and schizophrenia, attention deficit disorders (ADD), and attention deficit hyperactivity disorders (ADHD).

3. The method according to claim 1, wherein the CNS disorder is migraine.

4. The method according to claim 1, wherein the non-caffeine therapeutic agent is selected from the group consisting of a beta-blocker, adrenergic agonist, adrenergic antagonist, calcium channel blocker, antiepileptic medication, tricyclic antidepressant, selective serotonin reuptake inhibitor, methysergide maleate, analgesic, non-steroidal anti-inflammatory drug (NSAID), serotonin receptor agonist, and ergot derivative.

5. The method according to claim 4, wherein the calcium channel blocker is verapamil or amlodipine.

6. The method according to claim 4, wherein the antiepileptic medication is valproic acid, topiramate, or gabapentin.

7. The method according to claim 4, wherein the tricyclic antidepressant is amitriptyline, nortriptyline, or desipramine.

8. The method according to claim 4, wherein the selective serotonin reuptake inhibitor is paroxetine, fluoxetine, duloxetine or sertraline.

9. The method according to claim 4, wherein the NSAID is selected from the group consisting of aspirin, acetaminophen, ibuprofen, flurbiprofen, ketoprofen, naproxen, oxaprozin, etodolac, indomethacin, ketorolac, nabumetane, piroxicam, celecoxib, rofecoxib, meloxicam, JTE-522, L-745,337, and NS398.

10. The method according to claim 4, wherein the serotonin rkeceptor agonist is sumatriptan, naratriptan, almotriptan, rizatriptan, eletriptan, frovatriptan or zolmitriptan

11. The method according to claim 4, wherein the ergot derivative is dihydroergotamine.

12. The method according to claim 4, wherein the beta-blocker is selected from the group consisting of acebutolol, atenolol, carvedilol, betaxolol, levobunolol, cartelol, metoprolol, nadolol, oxprenolol, pindolol, propranolol, sotalol, and timolol.

13. The method according to claim 1, wherein caffeine or the non-caffeine therapeutic agent is administered to said subject via a route selected from the group consisting of oral, buccal, sublingual, rectal, topical, transmucosal, percutaneous, implantable, parenteral, subcutaneous, intramuscular, intradermal, intravenous, intrathecal, intracranial, intraperitoneal, transdermal, intratracheal, intravaginal, endocervical, intrathecal, intranasal, intravesicular, intraocular, transaural, intravascular, and extravascular route of administration.

14. The method according to claim 1, wherein caffeine or the non-caffeine therapeutic agent is administered orally to said subject.

15. The method according to claim 14, wherein the amount of caffeine administered is 100 mg to 1500 mg per dose.

16. The method according to claim 14, wherein the amount of caffeine administered is 400 mg to 1200 mg per dose.

17. The method according to claim 14, wherein the non-caffeine therapeutic agent is propranolol, and the CNS disorder is migraine.

18. The method according to claim 17, wherein the amount of propranolol administered is 1 to 2000 mg per dose.

19. The method according to claim 17, wherein the amount of propranolol administered is 10 to 200 mg per dose.

20. The method according to claim 14, wherein the non-caffeine therapeutic agent is timolol, and the CNS disorder is migraine.

21. The method according to claim 20, wherein the amount of timolol administered is 0.1 mg to 1000 mg per dose.

22. The method according to claim, wherein the amount of timolol administered is 1 to 100 mg per dose.

23. A method of preventing or reducing the risk of developing a central nervous system (CNS) disorder, comprising: administering to the subject a therapeutically amount of caffeine in combination with another non-caffeine therapeutic agent to a person at risk of developing the CNS disorder.

24. The method according to claim 23, wherein the CNS disorder is selected from the group consisting of headache, epilepsy, pain, Parkinson's disease, psychiatric disorders such as anxiety, bipolar disorder, depression, and schizophrenia, attention deficit disorders (ADD), and attention deficit hyperactivity disorders (ADHD).

25. The method according to claim 23, wherein the CNS disorder is migraine.

26. The method according to claim 23, wherein the non-caffeine therapeutic agent is selected from the group consisting of a beta-blocker, adrenergic agonist, adrenergic antagonist, calcium channel blocker, antiepileptic medication, tricyclic antidepressant, selective serotonin reuptake inhibitor, methysergide maleate, analgesic, non-steroidal anti-inflammatory drug (NSAID), serotonin receptor agonist, and ergot derivative.

27. The method according to claim 26, wherein the beta-blocker is selected from the group consisting of acebutolol, atenolol, carvedilol, betaxolol, levobunolol, cartelol, metoprolol, nadolol, oxprenolol, pindolol, propranolol, sotalol, and timolol.

28. The method according to claim 23, wherein caffeine or the non-caffeine therapeutic agent is administered to said person via a route selected from the group consisting of oral, buccal, sublingual, rectal, topical, percutaneous, implantable, parenteral, subcutaneous, intramuscular, intradermal, intravenous, intrathecal, intracranial, intraperitoneal, transdermal, intratracheal, intravaginal, endocervical, intrathecal, intranasal, intravesicular, intraocular, transaural, intravascular, epidural & intraosseous and extravascular route of administration.

29. The method according to claim 23, wherein caffeine or the non-caffeine therapeutic agent is administered orally to said person.

30. The method according to claim 29, wherein the amount of caffeine is greater than 300 mg per dose.

31. The method according to claim 29, wherein the amount of caffeine administered is 100 mg to 1500 mg per dose.

32. The method according to claim 29, wherein the amount of caffeine administered is 400 mg to 1200 mg per dose.

33. The method according to claim 23, wherein the non-caffeine therapeutic agent is propranolol, and the CNS disorder is migraine.

34. The method according to claim 33, wherein the amount of propranolol administered is 1 to 2000 mg per dose.

35. The method according to claim 33, wherein the amount of propranolol administered is 10 to 200 mg per dose.

36. The method according to claim 23, wherein the non-caffeine therapeutic agent is timolol, and the CNS disorder is migraine.

37. The method according to claim 36, wherein the amount of timolol administered is 0.1 mg to 1000 mg per dose.

38. The method according to claim 36, wherein the amount of timolol administered is 1 to 100 mg per dose.

39. A pharmaceutical composition, comprising: caffeine at a dose greater than 5 mg/kg; and a non-caffeine therapeutic agent in a uniform dosage, wherein the non-caffeine therapeutic agent is selected from the group consisting of a beta-blocker, adrenergic agonist, adrenergic antagonist, calcium channel blocker, antiepileptic medication, tricyclic antidepressant, selective serotonin reuptake inhibitor, methysergide maleate, analgesic, non-steroidal anti-inflammatory drug (NSAID), selective serotonin/norepinephrine reuptake inhibitors, serotonin receptor agonist, and ergot derivative.

40. The pharmaceutical composition according to claim 39, wherein the calcium channel blocker is verapamil or amlodipine.

41. The pharmaceutical composition according to claim 39, wherein the antiepileptic medication is valproic acid, topiramate, or gabapentin.

42. The pharmaceutical composition according to claim 39, wherein the tricyclic antidepressant is amitriptyline, nortriptyline, or desipramine.

43. The pharmaceutical composition according to claim 39, wherein the selective serotonin reuptake inhibitor is paroxetine, fluoxetine, or sertraline.

44. The pharmaceutical composition according to claim 39, wherein the NSAID is selected from the group consisting of aspirin, acetaminophen, ibuprofen, flurbiprofen, ketoprofen, naproxen, oxaprozin, etodolac, indomethacin, ketorolac, nabumetane, piroxicam, celecoxib, rofecoxib, meloxicam, JTE-522, L-745,337, and NS398.

45. The pharmaceutical composition according to claim 39, wherein the serotonin receptor agonist is sumatriptan, naratriptan, almotriptan, rizatriptan, eletriptan, frovatriptan or zolmitriptan.

46. The pharmaceutical composition according to claim 39, wherein the ergot derivative is dihydroergotamine.

47. The pharmaceutical composition according to claim 39, wherein the beta-blocker is selected from the group consisting of acebutolol, atenolol, carvedilol, betaxolol, levobunolol, cartelol, metoprolol, nadolol, oxprenolol, pindolol, propranolol, sotalol, and timolol.

48. The pharmaceutical composition according to claim 39, wherein the composition is in an oral dosage form.

49. The pharmaceutical composition according to claim 39, wherein the oral dosage form is selected from the group consisting of tablets, suppositories, pills, capsules, powders, liquids, and liquid suspensions.

50. The pharmaceutical composition according to claim 39, wherein the amount of caffeine in the pharmaceutical composition is 100 mg to 1500 mg per dose.

51. The pharmaceutical composition according to claim 39, wherein the amount of caffeine in the pharmaceutical composition is 400 mg to 1200 mg per dose.

52. The pharmaceutical composition according to claim 39, wherein the non-caffeine therapeutic agent is propranolol.

53. The pharmaceutical composition according to claim 52, wherein the amount of propranolol in the composition is 1 to 2000 mg.

54. The pharmaceutical composition according to claim 52, wherein the amount of propranolol in the composition is 10 to 200 mg.

55. The pharmaceutical composition according to claim 52, wherein the amount of propranolol in the composition is 20 to 60 mg.

56. The pharmaceutical composition according to claim 52, wherein the ratio of caffeine to propranolol in the composition is ≧8.

57. The pharmaceutical composition according to claim 52, wherein the ratio of caffeine to propranolol in the composition is ≧10.

58. The pharmaceutical composition according to claim 52, wherein the ratio of caffeine to propranolol in the composition is ≧12.

59. The pharmaceutical composition according to claim 52, wherein the ratio of caffeine to propranolol in the composition is ≧15.

60. The pharmaceutical composition according to claim 39, wherein the non-caffeine therapeutic agent is timolol.

61. The pharmaceutical composition according to claim 60, wherein the amount of timolol in the composition is 0.1 mg to 1000 mg.

62. The pharmaceutical composition according to claim 60, wherein the amount of timolol in the composition is 1 to 100 mg.

63. A kit for treating or preventing a central nervous system (CNS) disorder in a subject in need of such treatment or at the risk of developing a CNS disorder, comprising: caffeine, and another non-caffeine therapeutic agent.

64. The kit according to claim 63, wherein caffeine and the non-caffeine therapeutic agent are contained in the same container.

65. The kit according to claim 64, wherein caffeine and the non-caffeine therapeutic agent are combined mixed in a uniform dosage.

66. The kit according to claim 63, wherein caffeine and the non-caffeine therapeutic agent are contained in the separate containers.

67. The kit according to claim 63, further comprising: instruction of how to use the kit for treating or preventing the CNS disorder.

68. The kit according to claim 63, wherein the CNS disorder is selected from the group consisting of headache, epilepsy, pain, Parkinson's disease, psychiatric disorders such as anxiety, bipolar disorder, depression, and schizophrenia, attention deficit disorders (ADD), and attention deficit hyperactivity disorders (ADHD).

69. The kit according to claim 63, wherein the CNS disorder is migraine.

70. A bi-layered table comprising an effective amount of caffeine and an effective amount of one or more active agents comprising beta-blocker, adrenergic agonist, adrenergic antagonist, calcium channel blocker, antiepileptic medication, tricyclic antidepressant, selective serotonin reuptake inhibitor, methysergide maleate, analgesic, non-steroidal anti-inflammatory drug (NSAID), serotonin receptor agonist, and ergot derivative, or a combination thereof.

71. A bi-layered tablet composition comprising:
a first immediate-release layer comprising beta blocker and caffeine;
a second controlled-release layer comprising beta blocker and caffeine, alternatively a therapeutically effective amount of a third agent; and
a pharmaceutically acceptable carrier.

72. A pharmaceutical composition comprising:
wherein said composition is capable of providing a therapeutically effective plasma concentration of caffeine and a beta blocker in about 1 minute to about 20 minutes, following oral administration; and alternatively a therapeutically effective amount of a third agent.

73. A pharmaceutical composition comprising:
caffeine, a beta-blocker in a relative weight ratio of a beta-blocker, caffeine, alternatively a third active agent at (1-3):(10-50):(1-3), respectively; and
a pharmaceutically acceptable carrier.

74. The composition of claims 70, 71, 72 or 73, wherein said third agent is selected form a group consisting of a beta-blocker, adrenergic agonist, adrenergic antagonist, calcium channel blocker, antiepileptic medication, tricyclic antidepressant, selective serotonin reuptake inhibitor, methysergide maleate, analgesic, non-steroidal anti-inflammatory drug (NSAID), serotonin receptor agonist, and ergot derivative.

75. The composition of claims 70, 71, 72 or 73, wherein said third agent is calcium channel blocker verapamil or amlodipine.

76. The composition of claims 70, 71, 72 or 73, wherein said third agent is valproic acid, topiramate, or gabapentin.

77. The composition of claims 70, 71, 72 or 73, wherein said third agent is amitriptyline, nortriptyline, or desipramine.

78. The composition of claims 70, 71, 72 or 73, wherein said third agent is paroxetine, fluoxetine, or sertraline.

79. The composition of claims 70, 71, 72, or 73, wherein said third agent is aspirin, acetaminophen, ibuprofen, flurbiprofen, ketoprofen, naproxen, oxaprozin, etodolac, indomethacin, ketorolac, nabumetane, piroxicam, celecoxib, rofecoxib, meloxicam, JTE-522, L-745,337, or NS398.

80. The composition of claims 70, 71, 72 or 73, wherein said third agent is sumatriptan, naratriptan, almotriptan, rizatriptan, eletriptan, frovatriptan or zolmitriptan.

81. The composition of claims 70, 71, 72 or 73, wherein said third agent is dihydroergotamine.

82. The composition of claims 70, 71, 72 or 73, wherein said third agent is acebutolol, atenolol, carvedilol, betaxolol, levobunolol, cartelol, metoprolol, nadolol, oxprenolol, pindolol, propranolol, sotalol, or timolol.

83. The composition of claims 70, 71, 72, or 73, wherein said caffeine is at a dose of greater than 300 mg.

84. The composition of claims 70, 71 or 72, wherein said beta blocker is propranolol.

85. The composition of claim 84, wherein said propranolol is present at about 40 to about 60 mg.

86. The composition of claim 83, wherein said dose is about 1000 mg.

87. A pharmaceutical composition, comprising: caffeine; and a beta-blocker, wherein said beta blocker is at a dose less than 80 mg.

88. The composition of claim 87, wherein said caffeine is at a dose of from about 400 mg to about 1000 mg.

89. The composition of 39, wherein said caffeine is at a dose of about 6 mg/kg.

90. A effervescent pharmaceutical composition comprising caffeine and one or more additional active agent comprising beta-blocker, adrenergic agonist, adrenergic antagonist, calcium channel blocker, antiepileptic medication, tricyclic antidepressant, selective serotonin reuptake inhibitor, methysergide maleate, analgesic, non-steroidal anti-inflammatory drug (NSAID), serotonin receptor agonist, and ergot derivative The composition of claim 88, wherein said one or more additional active agent comprises propranolol.

91. The composition of claim 90, wherein said caffeine is in a dose of about 400 mg to about 1000 mg.

92. The composition of claim 90, wherein said caffeine is in a dose greater than 5 mg/kg.

93. The composition of claim 90, wherein said caffeine is in a dose greater than 300 mg.

94. The composition of claim 91, wherein said propranolol is in a dose of less than 80 mg.

95. The composition of 92, wherein said one or more active agent is propranolol.

96. The composition of claim 90 or 95, wherein said propranolol is in a dose of about 40 mg to about 200 mg.
